(19) United States
(12) Patent Application Publication
(10) Pub. No.: US 2020/0000549 A1
(43) Pub. Date: Jan. 2, 2020

(54) DENTAL SURGICAL STENT

(71) Applicant: CORNERSTONE SPECIALTY PRODUCTS, LLC, Irvine, CA (US)

(72) Inventors: Hamid Reza ABEDI, Irvine, CA (US); Ajay KUMAR, Palmdale, CA (US); Mahmoud TORABINEJAD, Loma Linda, CA (US); Arin ALEXANDER, Porter Ranch, CA (US)

(73) Assignee: CORNERSTONE SPECIALTY PRODUCTS, LLC, Irvine, CA (US)

(21) Appl. No.: 16/024,354

(22) Filed: Jun. 29, 2018

(51) Int. Cl. A61C 1/08; A61C 19/00; A61C 5/40

(52) U.S. Cl. CPC A61C 1/082; A61B 17/176; A61C 5/40; A61C 19/00

(57) ABSTRACT

Dental surgical stents can be used to assist a dental surgeon. A hard tissue stent can include an access hole formed in one side and may be placed over one or more of the patient's teeth where the access hole is accurately aligned at a location for drilling to, for example, remove infection. Suction channels may be included in the stent to provide suction about the access hole to remove saliva, water, bone debris and the like, during the surgical procedure. A soft tissue stent may be placed over one or more of the patient's teeth, where the stent can include a mark positioned where the surgeon is to cut to create an access flap in the soft tissue. By keeping the flap attached to the semi-circular window of the soft tissue stent, the surgeon can more easily return the flap to the proper position.

### BACKGROUND OF THE INVENTION

### 1. Field of the Invention

Embodiments of the invention relates generally to dental tools. More particularly, the invention relates to dental surgical stents that fit about a patient's tooth or teeth to help guide a dental professional during a procedure, such as a root canal, apicoectomy, or the like.

### 2. Description of Prior Art and Related Information

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

The primary aim of any endodontic treatment is to disinfect the root canal system in order to reduce the bacterial load as much as possible and to seal the system to prevent ingress or egress of bacteria or their byproducts. A root canal procedure often accesses the root from the top of the tooth to remove infection therein. In some applications, a dental professional, such as an endodontist, would need to access infection of the root from the bottom of the tooth. A flap may be made of the soft tissue to provide access to the bone and a tool may be used to remove the bone to gain access to the area containing the infection, such as a root tip. The infection may be removed, and the flap replaced to heal. In some circumstances, the root tip may be removed, and the root canal treated and sealed.

Often, once a flap is cut, it is difficult to handle and maintain in its proper position during the surgical procedure. The surgeon needs to keep the flap out of the way during the surgery and properly position the flap back in place thereafter. The soft tissue flap may be difficult to handle after it is cut.

Moreover, to access the infection, the surgeon may need to remove a substantial amount of bone to not only gain access but also to visually ensure the position and depth of hard tissue is correct.

Finally, during these dental procedures, sufficient irrigation and proper suction is needed to remove water, saliva and the like, as well as suction to remove bone debris as the surgeon gains access to the infection.

In view of the foregoing, there is a need for dental surgical stents that may secure the flap during surgery as well as provide a hard tissue drilling location and permit suction during dental surgery.

### SUMMARY OF THE INVENTION

Embodiments of the present invention provide a dental surgical stent comprising a stent body configured to drape over at least one tooth, the stent body including side members, where at least one of the side members extends downward to cover at least a portion of a gum region of the at least one tooth; suction channels formed in the stent body, the suction channels terminating at openings in multiple locations in the stent body; and a suction connection fluidly connected to the suction channels, the suction connection configured to connect to a suction tube to draw a suction through the suction connection, through the suction channels and draw liquids and debris from the patient at the openings.

In some embodiments, the present invention provides a method of using a dental surgical stent comprising placing the stent body over the at least one or more teeth; drilling hard tissue through the access opening; and suctioning bone debris through the suction channels.

In some embodiments, the present invention provides a method of using the dental surgical stent comprising placing the stent body over the at least one or more teeth; cutting soft tissue along an incision line; flapping the soft tissue, while placed against an inside surface of a stent flap portion, outward away from a gum region of the patient; removing infection below the soft tissue; and reattaching a flap of the soft tissue by moving the stent flap portion to a position aligned with the stent body.

Embodiments of the present invention further provide a hard tissue stent comprising a stent body configured to drape over at least one tooth, the stent body including side members, where at least one of the side members extends downward to cover at least a portion of a gum region of the at least one tooth; an access opening formed on the side member of the stent body, the access opening positioned at a location for drilling hard tissue thereunder; suction channels formed in the stent body, at least a portion of the suction channels terminating at openings adjacent the access opening; and a suction connection fluidly connected to the suction channels, the suction connection configured to connect to a suction tube to draw a suction through the suction connection, through the suction channels and draw liquids and debris from the patient at the openings.

Embodiments of the present invention also provide a soft tissue stent comprising a stent body configured to drape over at least one tooth, the stent body including side members, where at least one of the side members extends downward to cover at least a portion of a gum region of the at least one tooth; and an incision line formed in the side member of the stent body; wherein the incision line is formed as an arc spanning about 180 degrees, the incision line positioned adjacent the gum region when the stent body is disposed over the at least one tooth of a patient.

In some embodiments, the incision line is pre-cut through the stent body.

In some embodiments, the soft tissue stent further comprises suction channels formed in the stent body, at least a portion of the suction channels terminating at openings adjacent the access opening; and a suction connection fluidly connected to the suction channels, the suction connection configured to connect to a suction tube to draw a suction through the suction connection, through the suction channels and draw liquids and debris from the patient at the openings.

In some embodiments, the hard tissue stent and the soft tissue stent may further include a backflow prevention device disposed adjacent the suction connection.

These and other features, aspects and advantages of the present invention will become better understood with reference to the following drawings, description and claims.

### BRIEF DESCRIPTION OF THE DRAWINGS

Some embodiments of the present invention are illustrated as an example and are not limited by the figures of the accompanying drawings, in which like references may indicate similar elements.

FIG. 1 illustrates a perspective view of a hard tissue stent positioned in a patient's mouth according to an exemplary embodiment of the present invention;

FIG. 2 illustrates a perspective view of the hard tissue stent of FIG. 1, positioned on the patent's teeth;

FIG. 3 illustrates a perspective view of the hard tissue stent of FIG. 1, showing movement of debris through the stent via suction;

FIG. 4 illustrates a dental tool used with the hard tissue stent of FIG. 1, showing bone debris being pulled through suction channels thereof;

FIG. 5 illustrates a soft tissue stent positioned over a patient's mouth and having an indicator for cutting a surgical flap therein;

FIG. 6 illustrates the cutting of a flap through the soft tissue stent of FIG. 5;

FIG. 7 illustrates the soft tissue stent of FIG. 5 retaining the flap while exposing an infection area;

FIG. 8 illustrates a dental tool removing infection from the infection area while the soft tissue stent of FIG. 5 retains the flap in a secured position; and

FIG. 9 illustrates surgical glue being applied to the flap for accurate reattachment through assistance of the soft tissue stent of FIG. 5.

Unless otherwise indicated illustrations in the figures are not necessarily drawn to scale.

The invention and its various embodiments can now be better understood by turning to the following detailed description wherein illustrated embodiments are described. It is to be expressly understood that the illustrated embodiments are set forth as examples and not by way of limitations on the invention as ultimately defined in the claims.

### DETAILED DESCRIPTION OF THE PREFERRED EMBODIMENTS AND BEST MODE OF INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

As is well known to those skilled in the art, many careful considerations and compromises typically must be made when designing for the optimal configuration of a commercial implementation of any system, and in particular, the embodiments of the present invention. A commercial implementation in accordance with the spirit and teachings of the present invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

Broadly, embodiments of the present invention provide dental surgical stents that can be used to assist a dental surgeon during surgeries. In one embodiment, a hard tissue stent can include an access hole formed in one side. The hard tissue stent may be placed over one or more of the patient's teeth where the access hole is accurately aligned at a location for drilling to gain appropriate access to, for example, remove infected areas. Suction channels may be included in the hard tissue stent to provide suction near and in the access hole to remove saliva, water, bone debris and the like, during the surgical procedure. A soft tissue stent may be placed over one or more of the patient's teeth, where the stent can include a mark positioned where the surgeon is to cut to create an access flap in the soft tissue. Typically, soft tissue flaps may be difficult to handle. However, by keeping the flap attached to the semi-circular window of the soft tissue stent, the surgeon can more easily return the flap to the proper position. The soft tissue stent, when used by itself, may include suction channels similar to those shown and described below with respect to the hard tissue stent.

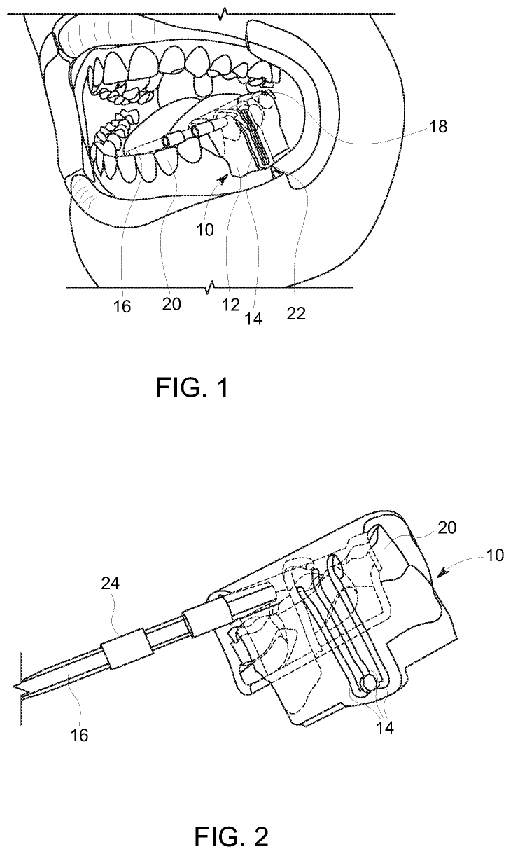

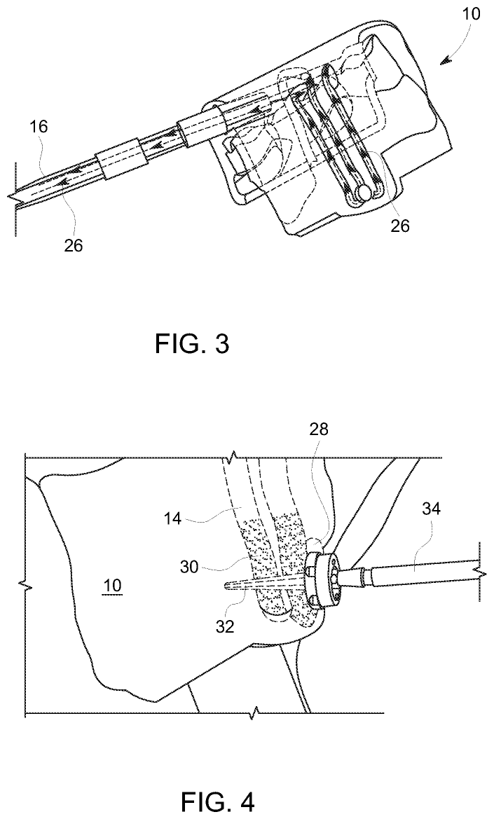

Referring now to FIGS. 1 through 4, a hard tissue stent 10 includes a body 12 configured to fit over one or more teeth 20 of a patient. Typically, the stent 10 may fit over the tooth below which there is a dental issue, such as an infection, and also fit over at least a portion of the teeth adjacent this tooth. The body 12 may extend down both the front, or buccal, and back, or lingual, sides of the tooth and gums.

One or more suction channels 14 may be formed within the body 12 to move saliva, water, bone debris, or the like, away from the stent 10, as shown by arrows 26, via suction applied by a suction connection 16. Typically, the suction channels 14 may terminate along the outer perimeter of an access opening 22 formed on one side of the body 12, as best shown in FIG. 2. In some embodiments, one or more suction channels 14 may terminate at sides of the body 12. In other embodiments, one or more opposite side suction channels 18 may create a suction on a side opposite the access opening 22.

A backflow prevention device 24 may be disposed between the stent 10 and the suction connection 16. The backflow prevention device 24 may be designed in various manners as may be known in the art. In some embodiments, the backflow prevention device 24 may be similar to those disclosed in U.S. Pat. No. 9,737,385, the contents of which are herein incorporated by reference.

As shown in FIG. 4, the access opening 22 may include a recessed or tapered portion 28 formed in the body 12 of the stent 10. The recessed or tapered portion 28 may be sized to receive a dental tool 34 therein, where a burr 32 of the dental tool 34 extends through the access opening 22 to remove bone, for example. When drilling is initiated, bone debris 30 may be taken up through the suction channels 14 as shown in FIG. 4.

The stent 10 may be custom designed for a particular patient, where a scan, such as an x-ray or CT scan, may be used to determine the precise position for the access opening 22. In other embodiments, the stent 10 may include a moldable interior material that may be pressed onto a patient's teeth to deform to the shape of the patient's tooth and to align the access opening 22 with a desired surgical location. The deformable material then be cured or made rigid or semi-rigid so that the access opening 22 is accurately placed and the stent 10 fits to the shape of the patient's teeth. Of course, other means to create the stent 10 as may be understood in the art are contemplated within the scope of the present invention.

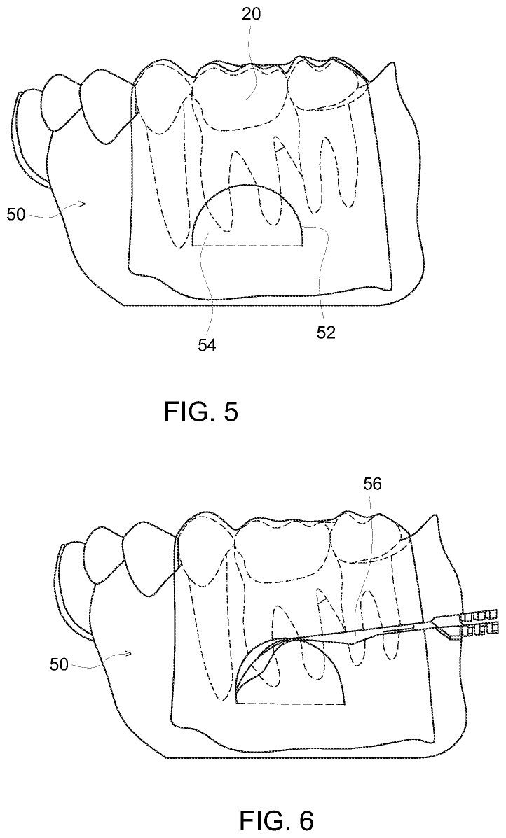

Referring now to FIGS. 5 through 9, a soft tissue stent 50 can include a stent flap 54 defined at least in part by an incision line 52. Typically, the incision line 52 is a semi-circular line, as shown, however other shapes may be contemplated within the scope of the present invention. Line the hard tissue stent 10, the soft tissue stent 50 may fit over one or more of the patient's teeth 20. While not shown in the Figures, the soft tissue stent 50 may also include suction channels and a suction connection as described in the hard tissue stent 10, described above. Sides of the soft tissue stent 50 may extend downward over the gums on at least one side thereof, typically on both the buccal and lingual sides of the gums.

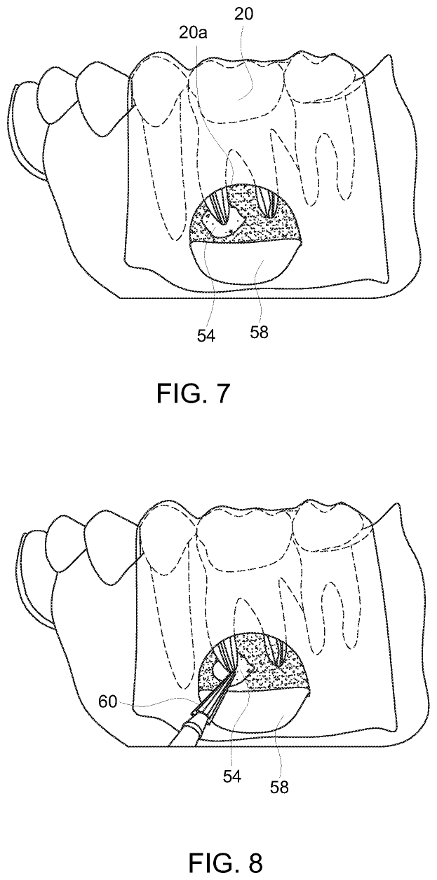

As shown in FIG. 6, the surgeon may use a tool, such as a scalpel 56, to cut along the incision line 52 of the soft tissue stent 50, cutting into the soft tissue to permit a soft tissue flap 58 to open while an exterior of the soft tissue flap 58 is supported by the stent flap 54, as shown in FIG. 7. With the soft tissue flap 58 open, the surgeon may have access to an infection region 54 formed at a root tip 20a.

As shown in FIG. 8, the surgeon may use a tool 60 to remove the infection region 54. Various techniques, as may be known in the art, may be used to ensure removal of all of the infection region 54. If irrigation is used, any suction channels formed in the stent 50 may help remove such irrigation, as well as infected tissue, saliva, or the like.

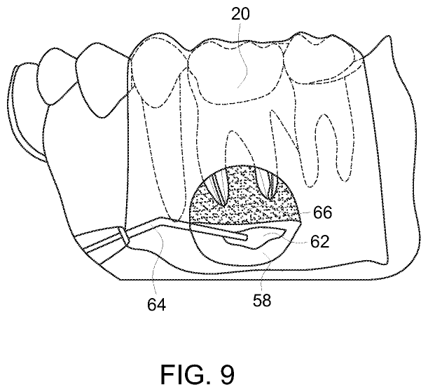

As shown in FIG. 9, once the infection is removed by the surgeon, tissue glue 62 may be applied to a back side of the soft tissue flap 58 with a tool 64. The soft tissue flap 58 may then be secured to the tissue 66 with the infection removed. By having the soft tissue flap 58 resting against the stent flap 54 during the procedure, the soft tissue flap 58 is easy to handle and kept in a position where accurate replacement is readily performed.

The incision line 52 defining the stent flap 54 may be designed for a particular patient based on scans, such as CT scans, or the patient. The incision line 52 may be pre-cut in the stent 50 or may be formed as a thinned material region, where the scalpel 56 may easily cut while cutting the soft tissue.

The suction channels 14 of the hard tissue stent 10 and the suction channels (not shown) of the soft tissue stent 50 may be designed in a similar manner to permit suction of saliva, water, blood, bone debris, infected tissue, or the like, away from the patient's mouth. While the Figures show one particular configuration, other configurations and designs are included within the scope of the present invention. The suction channels 14 may be optimized in size and shape to prevent clogging.

The stents 10, 50 may be formed of various materials as are known in the art. Typically, the stents 10, 50 may be formed from a polymeric material.

The stents 10, 50 may be used individually for specific procedures or, in some embodiments, the soft tissue stent 50 may be used to create a flap in the soft tissue while the hard tissue stent 10 may then be used to drill through hard tissue to access an infection area, root tip, or the like.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Many alterations and modifications may be made by those having ordinary skill in the art without departing from the spirit and scope of the invention. Therefore, it must be understood that the illustrated embodiments have been set forth only for the purposes of examples and that they should not be taken as limiting the invention as defined by the following claims. For example, notwithstanding the fact that the elements of a claim are set forth below in a certain combination, it must be expressly understood that the invention includes other combinations of fewer, more or different ones of the disclosed elements.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what incorporates the essential idea of the invention.

What is claimed is:

1. A dental surgical stent comprising:
a stent body configured to drape over at least one tooth, the stent body including side members, where at least one of the side members extends downward to cover at least a portion of a gum region of the at least one tooth;
suction channels formed in the stent body, the suction channels terminating at openings in multiple locations in the stent body; and
a suction connection fluidly connected to the suction channels, the suction connection configured to connect to a suction tube to draw a suction through the suction connection, through the suction channels and draw liquids and debris from the patient at the openings.

2. The dental surgical stent of claim 1, further comprising a backflow prevention device disposed adjacent the suction connection.

3. The dental surgical stent of claim 1, wherein the dental surgical stent is a hard tissue stent.

4. The dental surgical stent of claim 3, further comprising an access opening formed on the side member of the stent body, the access opening positioned at a location for drilling hard tissue thereunder.

5. The dental surgical stent of claim 4, further comprising a tapered region formed in the stent body surrounding the access opening.

6. A method of using the dental surgical stent of claim 4, wherein the method comprises:
placing the stent body over the at least one or more teeth;
drilling hard tissue through the access opening; and
suctioning bone debris through the suction channels.

7. The dental surgical stent of claim 1, wherein the dental surgical stent is a soft tissue stent.

8. The dental surgical stent of claim 7, further comprising an incision line formed along the side member of the stent body.

9. The dental surgical stent of claim 8, wherein the incision line is formed as an arc spanning about 180 degrees.

10. The dental surgical stent of claim 8, wherein the incision line is pre-cut through the stent body.

11. A method of using the dental surgical stent of claim 8, wherein the method comprises:
placing the stent body over the at least one or more teeth;
cutting soft tissue along the incision line;
flapping the soft tissue, while placed against an inside surface of a stent flap portion, outward away from a gum region of the patient;
removing infection below the soft tissue; and
reattaching a flap of the soft tissue by moving the stent flap portion to a position aligned with the stent body.

12. The method of claim 11, wherein surgical glue is used to reattach the flap of the soft tissue.

13. A hard tissue stent comprising:
a stent body configured to drape over at least one tooth, the stent body including side members, where at least one of the side members extends downward to cover at least a portion of a gum region of the at least one tooth;
an access opening formed on the side member of the stent body, the access opening positioned at a location for drilling hard tissue thereunder;
suction channels formed in the stent body, at least a portion of the suction channels terminating at openings adjacent the access opening; and
a suction connection fluidly connected to the suction channels, the suction connection configured to connect to a suction tube to draw a suction through the suction connection, through the suction channels and draw liquids and debris from the patient at the openings.

14. The hard tissue stent of claim 13, further comprising a backflow prevention device disposed adjacent the suction connection.

15. The hard tissue stent of claim 13, further comprising a tapered region formed in the stent body surrounding the access opening.

16. A soft tissue stent comprising:
a stent body configured to drape over at least one tooth, the stent body including side members, where at least one of the side members extends downward to cover at least a portion of a gum region of the at least one tooth;
an incision line formed in the side member of the stent body;
wherein the incision line is formed as an arc spanning about 180 degrees, the incision line positioned adjacent the gum region when the stent body is disposed over the at least one tooth of a patient.

17. The soft tissue stent of claim 16, wherein the incision line is pre-cut through the stent body.

18. The soft tissue stent of claim 16, further comprising:
suction channels formed in the stent body, at least a portion of the suction channels terminating at openings adjacent the access opening; and
a suction connection fluidly connected to the suction channels, the suction connection configured to connect to a suction tube to draw a suction through the suction connection, through the suction channels and draw liquids and debris from the patient at the openings.

19. The soft tissue stent of claim 18, further comprising a backflow prevention device disposed adjacent the suction connection.
